(19) United States
(12) Patent Application Publication
(10) Pub. No.: US 2016/0331398 A1
(43) Pub. Date: Nov. 17, 2016

(54) Vibratory Plunge Cutter

(71) Applicant: Cardiac Pacemakers, Inc., St. Paul, MN (US)

(72) Inventors: Arthur J. Foster, Blaine, MN (US); Jean M. Bobgan, Maple Grove, MN (US)

(21) Appl. No.: 15/151,234

(22) Filed: May 10, 2016

Related U.S. Application Data
(60) Provisional application No. 62/159,510, filed on May 11, 2015.

(51) Int. Cl. A61B 17/32; A61B 17/3211; A61N 1/05; A61B 17/3209

(52) U.S. Cl. CPC A61B 17/320068; A61B 17/32093; A61B 17/3211; A61N 1/0504

(57) ABSTRACT

Aspects of the present disclosure are directed toward apparatuses, methods, and systems that may include a blade comprising a leading point configured to puncture skin of a patient. In addition, the apparatuses, methods, and systems may also include a housing configured to hold the blade and provide a surface for a user to control the housing and the blade, the opening comprising sidewalls configured to control a depth to which the blade extends under the skin of the patient, and a motor arranged with the housing and configured to mechanically vibrate the blade.

### CROSS-REFERENCE TO RELATED APPLICATION

This application claims priority to Provisional Application No. 62/159,510, filed May 11, 2015, which is herein incorporated by reference in its entirety.

TECHNICAL FIELD

The present disclosure relates to medical devices and methods for creating an opening in a patient's skin for an implantable medical device. More specifically, the disclosure relates to devices and methods for facilitating implantation of the implantable medical device under the patient's skin in a minimally-invasive and efficient manner.

### BACKGROUND

Plunge cutters can be effective for providing a pocket underneath the skin of a patient for subcutaneous medical devices. Rather than applying a cutting motion as would be utilized with a scalpel, the tip of a plunge cutter, as opposed to an edge, can be pressed against the patient's skin. By applying a longitudinal force, along a body of the plunge cutter and perpendicular to the patient's skin, the tip can puncture the skin. Existing plunge cutters, however, can require high longitudinal forces in puncturing, which can result in inadequate pocket creation for the implantable medical device. Inadequate pocket creation can result in the device shifting within the body, and discomfort to the patient. Additionally, because existing plunge cutters have generally flat blades and the clinician typically pinches the patient's skin while cutting, the resulting incision typically is curved. Curved incisions can be more susceptible to reopening and allowing undesirable device movement.

### SUMMARY

In Example 1, an apparatus includes a blade comprising a leading point configured to puncture the skin of a patient, a housing having an opening through which the blade extends. The housing is configured to hold the blade and provide a surface for a user to control the housing and the blade. The opening includes sidewalls configured to control a depth to which the blade extends under the skin of the patient. The apparatus also includes a motor arranged with the housing and configured to mechanically vibrate the blade.

In Example 2, the apparatus according to Example 1, wherein the blade is symmetric about the leading point.

In Example 3, the apparatus according to Examples 1 or 2, wherein the blade comprises edges, symmetric about the leading point, configured to control a width at which the blade opens the skin of the patient.

In Example 4, the apparatus according to Example 3, wherein the edges are curved about a leading edge of the blade.

In Example 5, the apparatus according to Example 4, wherein the radius of curvature of the edges is approximately 90 degrees longitudinal from a horizontal plane.

In Example 6, the apparatus according to any of Examples 3-5, wherein the edges are at least one of serrated and beveled.

In Example 7, the apparatus according to any of Examples 1-6, further comprising a power source to power the motor to mechanically vibrate the blade.

In Example 8, the apparatus according to Example 7, wherein the power source is configured to activate in response to the user gripping the housing.

In Example 9, the apparatus according to Example 7 or Example 8, wherein the power source is configured to deactivate in response to the user releasing the housing.

In Example 10, the apparatus according to any of Examples 7-9, wherein the power source is arranged within the housing.

In Example 11, the apparatus according to any of Examples 7-10, wherein the power source comprises at least two modes that provide at least two levels of power to mechanically vibrate the blade.

In Example 12, the apparatus of according to Example 11, further comprising at least one control mechanism configured to switch between the at least two modes that provide the at least two levels of power to mechanically vibrate the blade.

In Example 13, the apparatus of according to any of Examples 1-12, wherein the motor is arranged within the housing, and the motor is directly coupled to the blade.

In Example 14, the apparatus of according to any of Examples 1-13, wherein the motor is at least one of a piezoelectric motor, an electromagnetic vibratory motor, an electrostatic vibratory motor, and an electroactive polymer.

In Example 15, the apparatus according to any of Examples 1-14, wherein the blade is coupled to the housing, and the motor is configured to mechanically vibrate the blade by transmitting vibrational energy through the housing.

In Example 16, an apparatus comprising a blade comprising a leading point configured to puncture skin of a patient; a housing comprising an opening through which the blade extends, the housing being configured to hold the blade and provide a surface for a user to control the housing and the blade, the opening comprising sidewalls configured to control a depth to which the blade extends under the skin of the patient; and a motor arranged with the housing and configured to mechanically vibrate the blade.

In Example 17, the apparatus of Example 16, wherein the blade is symmetric about the leading point.

In Example 18, the apparatus of Example 17, wherein the blade comprises edges, symmetric about the leading point, configured to control a width at which the blade opens the skin of the patient.

In Example 19, the apparatus of Example 16, further comprising a power source to power the motor to mechanically vibrate the blade.

In Example 20, the apparatus of Example 19, wherein the power source is configured to activate in response to the user gripping the housing.

In Example 21, the apparatus of Example 16, wherein the motor is at least one of a piezoelectric motor, an electromagnetic vibratory motor, an electrostatic vibratory motor, and an electroactive polymer.

In Example 22, the apparatus of Example 16, further comprising at least one control mechanism configured to switch between at least two modes that provide the at least two levels of power to mechanically vibrate the blade.

In Example 23, a method comprising: activating a motor, arranged with a housing, to mechanically vibrate a blade held within the housing; pressing a leading point of the blade against skin of a patient; puncturing the skin of the patient with the leading point of the blade; and extending the blade into the patient until sidewalls of the housing are against the skin of the patient.

In Example 24, the method of Example 23, wherein the step of activating the motor comprises activating one of at least two modes to provide one of at least two levels of power to mechanically vibrate the blade, and wherein the step of extending the blade into the patient comprises forming a pocket within the patient defined by a width of the blade.

In Example 25, the method of Example 23, further comprising removing the blade and inserting an implantable medical device, via an introducer, within the pocket defined by insertion of the blade into the patient, and closing the pocket to seal the implantable medical device within the patient.

In Example 26, the method of Example 24, wherein the step of closing the pocket comprises sealing the implantable medical device within the patient by at least one of suturing and gluing a cut in the skin of the patient.

In Example 27, a system comprising: an implantable medical device; an implantation apparatus comprising: a blade comprising a leading point configured to puncture the skin of a patient, a housing comprising an opening through which the blade extends, the housing being configured to hold the blade and provide a surface for a user to control the housing and the blade, the opening comprising sidewalls configured to control a depth to which the blade extends under the skin of the patient, and a motor arranged with the housing and configured to mechanically vibrate the blade; and an introducer configured to position the implantable medical device under the skin of the patient.

In Example 28, the system of Example 27, wherein the implantable medical device is at least one of an implantable loop recorder, a cardiac monitor, a pacemaker, and a defibrillator.

In Example 29, the system of Example 27, wherein the blade is symmetric about the leading point.

In Example 30, the system of Example 29, wherein the blade comprises edges, symmetric about the leading point, configured to control a width at which the blade opens the skin of the patient.

In Example 31, the system of Example 27, wherein the implantation apparatus further comprises at least one control mechanism configured to switch between at least two modes that provide the at least two levels of power to mechanically vibrate the blade.

In Example 32, the system of Example 31, further comprising a power source to power the motor to mechanically vibrate the blade.

In Example 33, the system of Example 32, wherein the power source is configured to activate in response to the user gripping the housing.

In Example 34, the system of Example 32, wherein the power source is configured to activate in response to the user pressing the at least one control mechanism.

In Example 35, the system of Example 27, wherein the motor is arranged within the housing, and the motor is directly coupled to the blade.

In Example 36, an apparatus comprises a blade comprising a leading point configured to puncture skin of a patient, the blade comprising an upper surface, a lower surface, and a cutting edge, wherein the blade is curved such that the lower surface is concave away from a plane that is tangent to a point at which an axis of symmetry is perpendicular to the upper surface, wherein the axis of symmetry extends between the upper surface and the lower surface; and a housing configured to hold the blade and provide a surface for a user to control the housing and the blade.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an exemplary apparatus for facilitating implantation of an implantable medical device in accordance with embodiments of the present invention.

FIGS. 2A-2Q show exemplary blades usable with an apparatus for facilitating implantation of an implantable medical device in accordance with embodiments of the present invention.

FIG. 3 shows an exemplary system including an implantation apparatus for implanting an implantable medical device, and an introducer in accordance with embodiments of the present invention.

FIG. 4 shows exemplary internal components of an apparatus for facilitating implantation of an implantable medical device in accordance with embodiments of the present invention.

FIG. 5 is a flow diagram depicting an exemplary method of implanting an implantable medical device in accordance with embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

### DETAILED DESCRIPTION

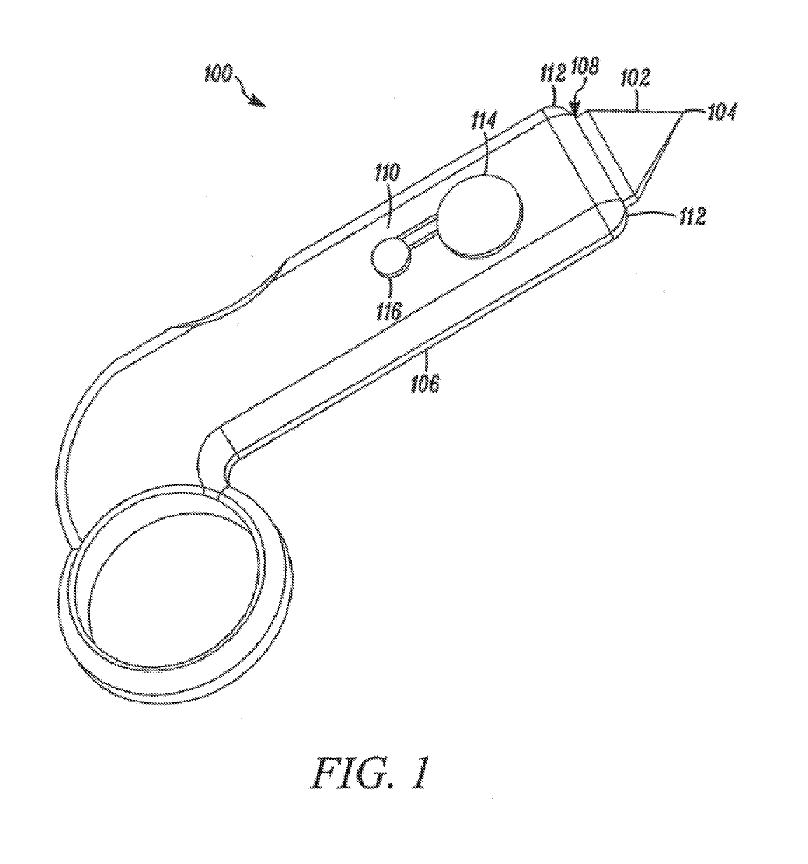

FIG. 1 shows an exemplary apparatus 100 for facilitating implantation of an implantable medical device in accordance with embodiments of the present invention. The apparatus 100 may include a blade 102 having a leading point 104. The leading point 104 of the blade 102 is configured to puncture the skin of a patient. The apparatus 100 also includes a housing 106. The housing 106 includes an opening 108 through which the blade 102 extends. An external surface 110 of the housing 106 may allow the user to control the housing 106 and the blade 102. In addition, the housing 106 may include sidewalls 112, which are configured to control a depth to which the blade 102 extends under the skin of the patient. The sidewalls 112 may control the depth to which the blade 102 extends under the skin of the patient by providing a stop or barrier such that the sidewalls 112 abut the skin of the patient, and indicates to the user controlling the apparatus 100 that the appropriate depth has been received.

In embodiments, the housing 110 may also be provided with a motor 114. The motor 114 may be configured to mechanically vibrate the blade 102. In this manner, the blade 102 may reduce the mechanical force needed to puncture the skin of the patient. In embodiments, such as shown in FIG. 1, the motor 114 is arranged on an external portion of the housing 110. In embodiments, the motor 114 may be arranged partially or completely within the housing. The motor 114 may be a piezoelectric motor, an electromagnetic vibratory motor, an electrostatic vibratory motor, or an electroactive polymer, and/or the like. The motor 114 may be powered by a power source (not shown) such as a battery. In embodiments, the motor 114 may be configured to vibrate the blade 102 directly and/or indirectly. For example, the motor 114 may be coupled directly to the blade, indirectly mechanically coupled to the blade, and/or the like. For example, although the blade 102 extends through the opening 108 in the housing 110, the blade 102 may also be coupled to the housing 110. In this manner, the motor 114 may be configured to mechanically vibrate the blade 102 by transmitting vibrational energy through the housing 106.

In addition, in embodiments, the apparatus 100 may include a control mechanism or switch 116 that is configured power on the motor 114. The control mechanism or switch 116 may switch the motor 114 on and off. In addition the control mechanism or switch 116 may switch the motor 114 to more than one mode (e.g., two, three, four) that provide more than one level of power to mechanically vibrate the blade 102. The different levels of power may provide multiple different speeds or frequencies at which the blade 102 is vibrated.

The illustrative apparatus 100 shown in FIG. 1 is not intended to suggest any limitation as to the scope of use or functionality of embodiments of the present invention. Neither should the illustrative apparatus 100 be interpreted as having any dependency or requirement related to any single component or combination of components illustrated therein. Additionally, any one or more of the components depicted in FIG. 1 may be, in embodiments, integrated with various ones of the other components depicted therein (and/or components not illustrated), all of which are considered to be within the ambit of the present invention. For example, the apparatus 100 may include more than one blade 102, the blade 102 may be integrated with the housing 106, the motor 114 may be integrated with the blade 102, and/or the like.

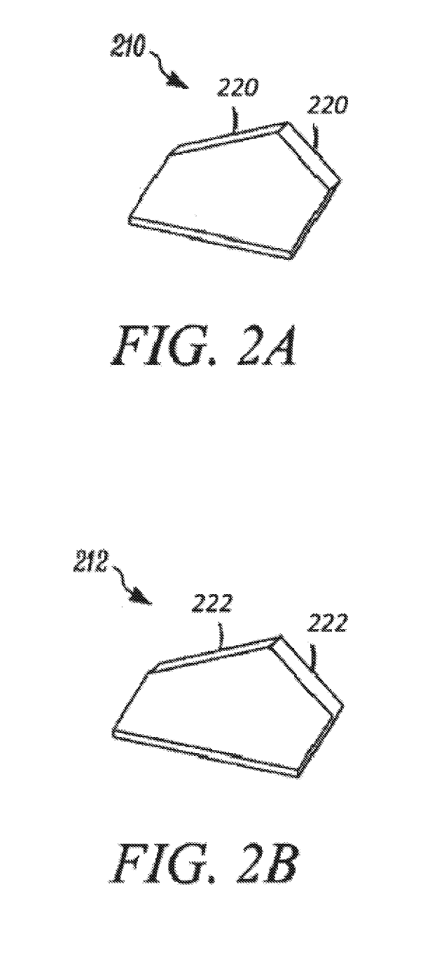

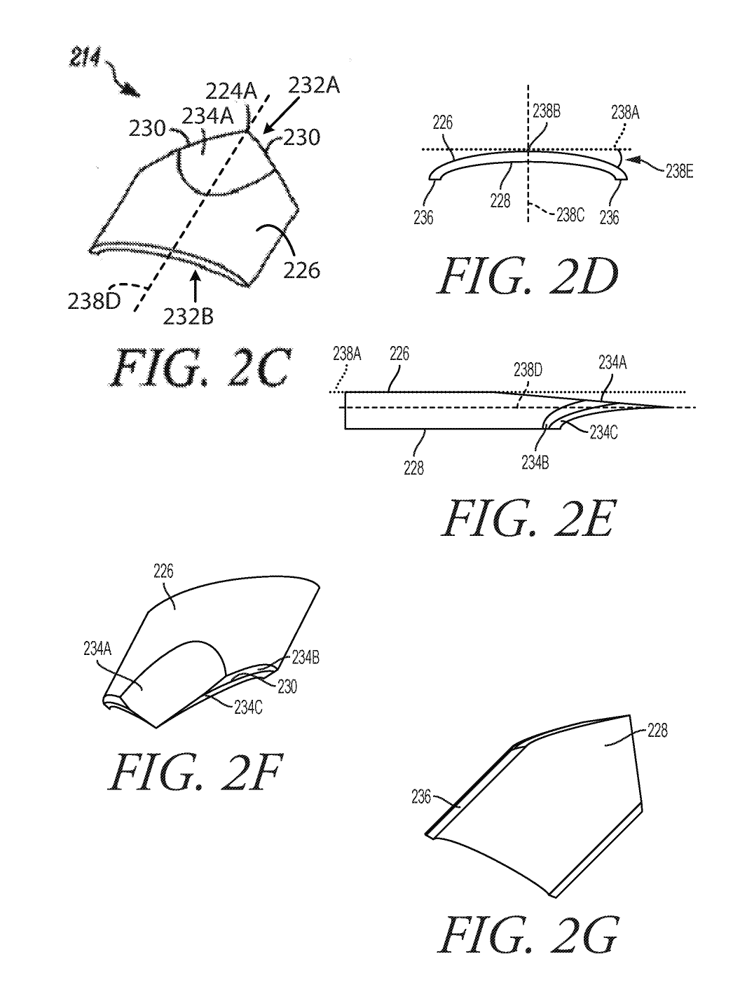

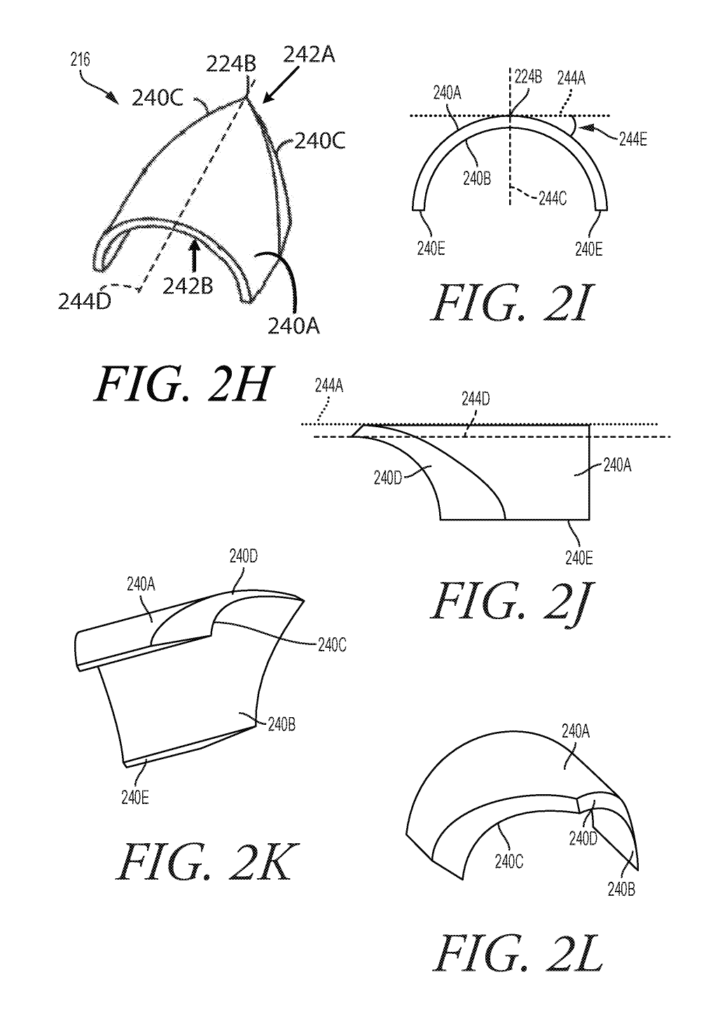

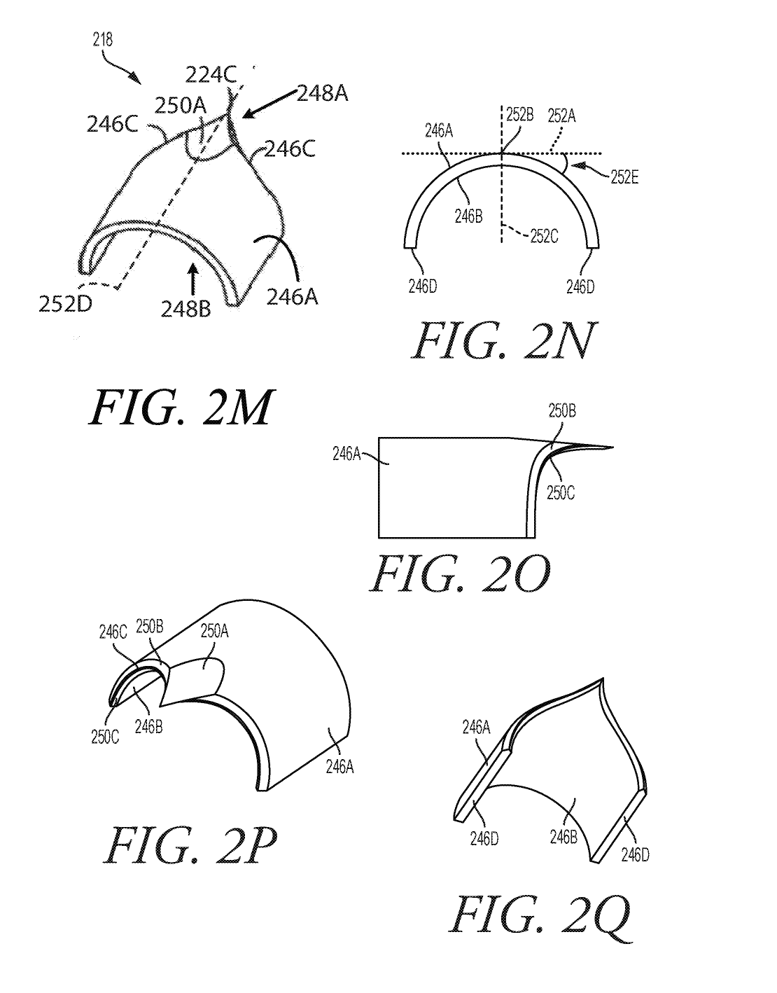

FIGS. 2A-2Q show exemplary blades usable with an apparatus for facilitating implantation of an implantable medical device in accordance with embodiments of the present invention. In embodiments, the apparatus may be, include, or be similar to the apparatus 100 depicted in FIG. 1. In embodiments, the apparatus may include a vibrating feature, as described above. In other embodiments, the apparatus may not include a vibrating feature. In embodiments, one or more blades may be provided with the apparatus and may, for example, be interchangeable to accommodate for differing patient characteristics.

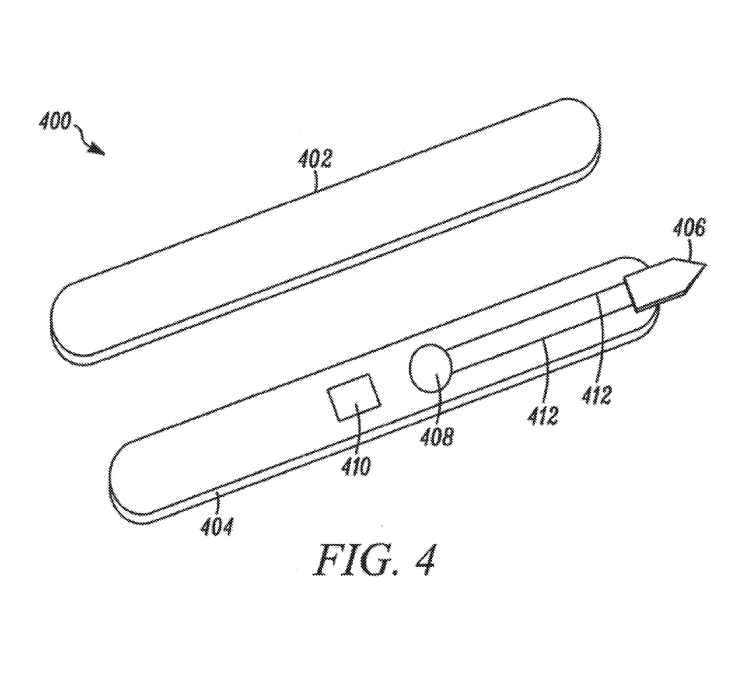

According to embodiments, any one or more of the blades 210, 212, 214, 216, and 218 may be symmetric about a leading point (e.g., the leading point 104 depicted in FIG. 4). FIGS. 2A and 2B depict embodiments of flat blades 210 and 212 in accordance with embodiments of the invention. As is shown in FIG. 2A, the blade 210 may include edges 220 that are configured to control a width at which the blade 210 opens the skin of the patient. For instance, the blade 212 shown in FIG. 2B has a larger width than the blade 210 shown in FIG. 2A. Thus, the edges 222 of the blade 212 shown in FIG. 2B open the skin of a patient to a larger width than the edges 220 of the blade 210 shown in FIG. 2A. In some embodiments, as is shown, for example, in FIGS. 2A and 2B, the edges 220, 222 of blade 210 and/or 212, respectively, may be beveled. In embodiments, the blades may have serrated edges. The blades may have beveled edges that are curved. Further, the blades may have bevels that have a wider thickness or depth than, for example, is shown in FIGS. 2A-2Q. The bevels may also be on a bottom surface of the blades as opposed to the top surface, for example, as shown in FIGS. 2A-2C.

Generally, when facilitating implantation of a medical device using a plunge cutter, according to embodiments of the invention, the clinician pinches the patient's skin at or near the implantation site, and plunges the blade into the portion of the patient's skin that is held between the clinician's pinching fingers. In many instances, when the clinician releases the skin, the resulting incision, formed by a flat blade of the plunge cutter, is curved. Curved incisions may be more likely to reopen, which may result in the implanted medical device migrating out of the patient's body or shifting in position in the patient's body. Reopened incisions may also be more susceptible to infection and/or other complications.

Accordingly, in embodiments, the blade of the plunge cutter may be curved such that, when the patient's skin is pinched and the blade inserted, the resulting incision (after the clinician releases the pinched skin) is substantially straight (that is, straight or nearly straight, allowing for variations due to skin geometry, inconsistent pinch sizes, and/or the like). In this manner, a curved blade may facilitate reduction of reopening incisions, device migration, and/or the like. FIGS. 2C-2Q depict embodiments of curved blades.

The illustrative blades 214, 216, and 218, shown in FIGS. 2C-2Q are not intended to suggest any limitation as to the scope of use or functionality of embodiments of the present invention. Neither should the illustrative blades 214, 216, and 218 be interpreted as having any dependency or requirement related to any single component, feature, or combination of components or features illustrated therein. Additionally, any one or more of the components or features depicted in FIGS. 2A-2P may be, in embodiments, integrated with various ones of the other components and/or features depicted therein (and/or components and/or features not illustrated), all of which are considered to be within the ambit of the present invention.

FIGS. 2C-2G depict an illustrative blade 214, in accordance with embodiments of the invention. As shown, the blade 214 includes a leading point 224A, an upper surface 226 and a lower surface 228. As the term is used herein, and throughout this disclosure, “upper surface” refers to a surface of a blade that is at least partially convex (e.g., has a positive curvature), while the term “lower surface” refers to a surface of a blade that is at least partially concave (e.g., has a negative curvature). A cutting edge 230 is disposed at a distal portion 232 of the blade 214. As shown, the blade 214 may include a first beveled surface 234A extending proximally between the leading point 224A and the upper surface 226. In embodiments, the first beveled surface 234A may be configured to be of any number of different sizes and/or shapes. Additionally, the distal portion 232A of the blade 214 may include a second beveled surface 234B bounded by the upper surface 226, the first beveled surface 234A, the cutting edge 230, and an outer edge 236. In embodiments, as shown in FIGS. 2C-2G, the blade 214 may include a second beveled surface 236 on each side of the leading point 224. As is also shown in FIGS. 2C-2G, the blade 214 may include a third beveled surface 234C, bounded by the leading point 224, the cutting edge 230, the outer edge 236, and the lower surface 228.

As is illustrated in FIGS. 2C-2E, the blade 214 may be curved such that the lower surface 228 is “concave away” from a plane 238A that is tangent to the upper surface 226. For example, in embodiments, the plane 238A may be tangent to an uppermost point on the upper surface 228, a center point on the upper surface 228, and/or the like. As shown, the plane 238A may be tangent to a point 238B at which a first symmetrical axis 238C, extending through the upper surface 226 and the lower surface 228, is perpendicular to the upper surface 226. In embodiments, the plane 238A may be orthogonal to a plane (not shown) that includes the point 238B and a second symmetrical axis 238D that extends from the leading point 224A to the proximal portion 232B of the blade 214. According to embodiments, an angle 238E defined between the upper surface 226 and the plane 238A may be configured to increase from zero degrees at the point 238B to approximately ninety degrees where the upper surface 226 engages the outer edge 236. In embodiments, the curvature may be designed such that the maximum measurement of the angle 238E is less than ninety degrees, while in other embodiments, the curvature may be designed such that the maximum measurement of the angle 238E is greater than ninety degrees.

FIGS. 2H-2L depict an illustrative blade 216, in accordance with embodiments of the invention. As shown, the blade 216 includes a leading point 224B, an upper surface 240A and a lower surface 240B. A cutting edge 240C is disposed at a distal portion 242A of the blade 216. As shown, the blade 216 may include a beveled surface 240D bounded by the upper surface 240A, the lower surface 240B, the cutting edge 240C, and an outer edge 240E.

As is illustrated in FIGS. 2H-2J, the blade 216 may be curved such that the lower surface 240B is “concave away” from a plane 244A that is tangent to the upper surface 240A. For example, in embodiments, the plane 244A may be tangent to an uppermost point on the upper surface 240A, a center point on the upper surface 240A, and/or the like. As shown, the plane 244A may be tangent to a point 244B at which a first symmetrical axis 244C, extending through the upper surface 240A and the lower surface 240B, is perpendicular to the upper surface 240A. In embodiments, the plane 244A may be orthogonal to a plane (not shown) that includes the point 244B and a second symmetrical axis 244D that extends from the leading point 224B to the proximal portion 242B of the blade 216. According to embodiments, an angle 244E defined between the upper surface 240A and the plane 244A may be configured to increase from zero degrees at the point 244B to approximately ninety degrees where the upper surface 240A engages the outer edge 240E. In embodiments, the curvature may be designed such that the maximum measurement of the angle 244E is less than ninety degrees, while in other embodiments, the curvature may be designed such that the maximum measurement of the angle 244E is greater than ninety degrees.

FIGS. 2M-2Q depict an illustrative blade 218, in accordance with embodiments of the invention. As shown, the blade includes a leading point 224C, an upper surface 246A and a lower surface 246B. A cutting edge 246C is disposed at a distal portion 248A of the blade 218. As shown, the blade 218 may include a first beveled surface 250A extending proximally between the leading point 224C and the upper surface 246A. In embodiments, the first beveled surface 250A may be configured to be of any number of different sizes and/or shapes. Additionally, the distal portion 248A of the blade 218 may include a second beveled surface 250B bounded by the upper surface 246A, the first beveled surface 250A, the cutting edge 246C, and an outer edge 246D. In embodiments, as shown, the blade 218 may include a second beveled surface 250B on each side of the leading point 224C. As is also shown, the blade 218 may include a third beveled surface 250C, bounded by the leading point 224C, the cutting edge 246C, the outer edge 246D, and the lower surface 246B.

As is illustrated in FIGS. 2M-2O, the blade 218 may be curved such that the lower surface 246B is “concave away” from a plane 252A that is tangent to the upper surface 246A. For example, in embodiments, the plane 252A may be tangent to an uppermost point on the upper surface 246A, a center point on the upper surface 246A, and/or the like. As shown, the plane 252A may be tangent to a point 252B at which a first symmetrical axis 252C, extending through the upper surface 246A and the lower surface 246B, is perpendicular to the upper surface 246A. In embodiments, the plane 252A may be orthogonal to a plane (not shown) that includes the point 252B and a second symmetrical axis 252D that extends from the leading point 224C to the proximal portion 248B of the blade 218. According to embodiments, an angle 252E defined between the upper surface 246A and the plane 252A may be configured to increase from zero degrees at the point 252B to approximately ninety degrees where the upper surface 246A engages the outside edge 246D. In embodiments, the curvature may be designed such that the maximum measurement of the angle 252E is less than ninety degrees, while in other embodiments, the curvature may be designed such that the maximum measurement of the angle 252E is greater than ninety degrees.

As noted above, FIGS. 2A-2Q show exemplary blades that are usable with apparatuses for facilitating implantation of an implantable medical device. In certain instances, the apparatuses may include a vibrating feature, as described above, for example, with reference to FIG. 1. The vibrating feature may be coupled to a blade and limit vibration of the blade in a particular direction. For example, the vibrating feature may vibrate the blade at least approximately along and/or at least approximately perpendicular to, an axis that extends from a leading point. The vibrating feature may vibrate the blade in any number of directions such that the direction of the motion of the blade is at least approximately parallel to a plane in which the axis that extends from the leading point lies. For example, the vibrating feature may vibrate blade 216 along the second symmetrical axis 244D (as shown in FIG. 2H) and/or in a direction that is at least approximately parallel to the plane 244A (as shown in FIG. 21). Similarly, the vibrating feature may vibrate blade 218 along the second symmetrical axis 252D (as shown in FIG. 2M) and/or in a direction that is at least approximately parallel to the plane 252A (as shown in FIG. 2N). Other blades discussed herein may be similarly limited in vibration by way of vibrating features coupled thereto.

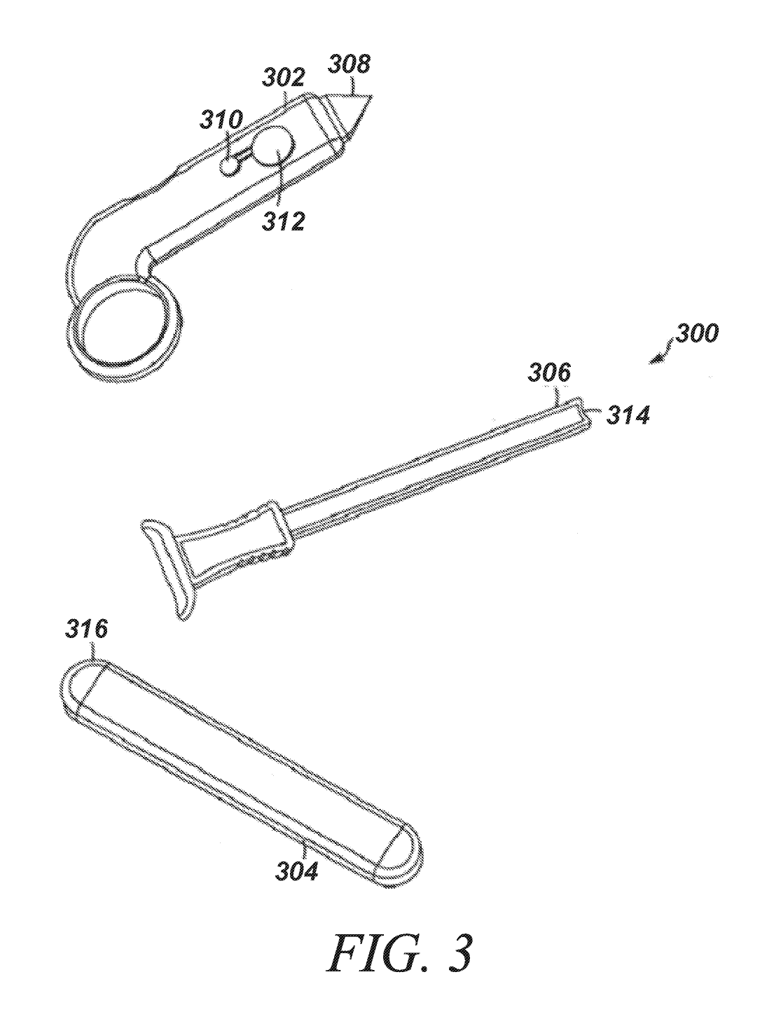

FIG. 3 shows an exemplary system 300 including an implantation apparatus 302 for facilitating implantation of an implantable medical device 304, and an introducer 306 that may be used in relation to embodiments of the present invention. The implantation apparatus 302, the implantable medical device 304, and the introducer 306 may be provided to a physician or surgeon as a package or kit. Thus, after the implantable medical device 304 is inserted under the skin of a patient, both the implantation apparatus 302 and the introducer 306 may be discarded. The package or kit may be sterile such that each of the implantation apparatus 302, the implantable medical device 304, and the introducer 306 are immediately useable by a physician or surgeon.

As described in further detail with reference to, for example, FIG. 1, the implantation apparatus 302 includes a blade 308 configured to puncture skin of a patient, a housing 310 from which the blade extends, and a motor 312 configured to mechanically vibrate the blade. The motor 312 may also be provided internal to the housing 310. The implantable medical device 304 may be an implantable loop recorder, a cardiac monitor, a pacemaker, a defibrillator, a drug-delivery device, a neuro-stimulation device, and/or any other type of implantable medical device. The width of the blade 308 may be configured to create an incision large enough to allow for implantation of the implantable medical device 304. For example, the width of the blade 308 may be configured based on one or more dimensions (e.g., width, length, thickness, etc.) of the implantable medical device 304. In this manner, the blade 308 may create a pocket underneath the skin of the patient having a width that complements the width of the implantable medical device 304.

The introducer 306 is configured to position the implantable medical device 304 under the skin of the patient. The introducer 306 may also include a surface 314 that interfaces with an end portion 316 of the implantable medical device 304. The surface 314 of the introducer 306 may complement the end portion 316 of the implantable medical device 304 and thereby aid in insertion of the implantable medical device 304 under the skin of the patient.

FIG. 4 shows exemplary internal components of an apparatus 400 for facilitating implantation of an implantable medical device in accordance with embodiments of the present invention. The apparatus 400 may include a housing formed by an upper portion 402 and a lower portion 404. The upper portion 402 and the lower portion 404 are secured together to form the housing that may be manipulated and controllable by a user. In addition, internal components may be provided and secured with the upper portion 402 and/or the lower portion 404. Although internal components as shown as provided with the lower portion 404 in FIG. 4, the internal components may be similarly affixed with the upper portion 402, as a result of securing the upper portion 402 with the lower portion 404, and/or the like. In addition, after securement of the upper portion 402 and the lower portion 404, an opening (not shown) is formed through which a blade 406 extends. The opening may be formed, for example, by provided notches in one or more of the upper portion 402 and the lower portion 404. In addition, the opening may be formed by molding the one or more of the upper portion 402 and the lower portion 404 to include a space for the blade 406.

As is shown in FIG. 4, a motor 408 may be provided with the lower portion 404, and may be configured to vibrate the blade 406. The motor 408 may be a piezoelectric motor, an electromagnetic vibratory motor, an electrostatic vibratory motor, an electroactive polymer, and/or the like. In the illustrated embodiments, a power source 410 is arranged with the lower portion 404 such that it is within the housing when the upper portion 402 and the lower portion 404 are secured together. The motor 406 may be powered via the power source 410. The motor 408 may be coupled to the housing (e.g., indirectly coupled to the blade 406 via the housing), directly coupled to the blade 406 (e.g., integrated with the blade, welded to the blade, and/or the like), directly coupled to the blade 406 via one or more structural elements 412, and/or the like. The one or more structural elements 412 may be wires, bars, plates, or ribbons, and/or the like. As a result, vibration provided by the motor 408 may be directly provided to the blade 406 via the one or more structural elements 412. In other words, the motor 408 may vibrate the blade 406 via the one or more structural elements 412, as opposed to, or in addition to, vibrating the blade 406 via the housing.

In addition, the power source 410 may be configured to activate in response to a user gripping the housing. Further, the power source 410 may also be configured to deactivate in response to the user releasing the housing. In embodiments, the power source 410 may provide two (or more) modes that provide two (or more) levels of power to mechanically vibrate the blade 406. The two or more modes may be adjusted by the user applying pressure to the housing such that one squeeze, for example, may switch between the motor 408 on and off. In embodiments, squeezing the housing a second time may switch the motor 408 to a second level of vibration (speed or frequency of the vibration at which the blade 406 vibrates increases). A third squeeze of the housing may switch the motor 408 to a third (higher) level of vibration, and a fourth squeeze of the housing may switch the motor 408 off. In addition, the power source 410 may provide two (or more) modes that provide two (or more) levels of power to mechanically vibrate the blade 406 in response to a control mechanism or switch, for example as shown and discussed above with reference to FIG. 1, provided on an external surface of the upper portion 402 and/or the lower portion 404 of the housing. The control mechanism or switch may be a pressure switch, button, and/or another type of physical structure that may be actuateable by the user.

The illustrative apparatus 400 shown in FIG. 4 is not intended to suggest any limitation as to the scope of use or functionality of embodiments of the present invention. Neither should the illustrative apparatus 400 be interpreted as having any dependency or requirement related to any single component or combination of components illustrated therein. Additionally, any one or more of the components depicted in FIG. 4 may be, in embodiments, integrated with various ones of the other components depicted therein (and/or components not illustrated), all of which are considered to be within the ambit of the present invention. For example, the apparatus 400 may include more than one blade 406, the blade 406 may be integrated with the upper portion 402, the apparatus 400 may include more than one motor 408, the apparatus 400 may include more than one power source 410, and/or the like.

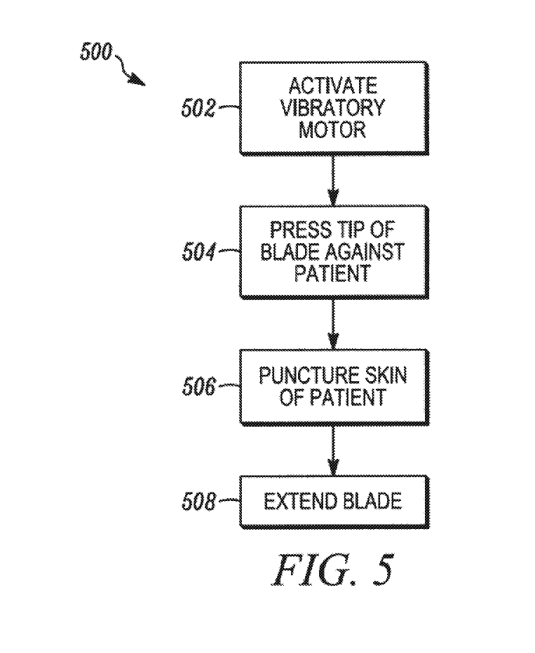

FIG. 5 is a flow diagram 500 depicting an exemplary method 500 of implanting an implantable medical device in accordance with embodiments of the present invention. The method 500 shown in FIG. 5 is operable with an apparatus, as discussed in detail herein, that may include a housing, a blade extending through an opening in the housing, and a vibratory motor configured to mechanically vibrate the blade. As is shown at block 502, the vibratory motor, arranged with the housing, is activated to mechanically vibrate the blade held within the housing. As noted above, the vibratory motor may be activated by engaging a switch (provided with the housing) or by a user gripping the housing. In addition, the vibratory motor may be activated in one or more modes. As described in detail above, the different modes may provide varying levels of power to mechanically vibrate the blade at different frequencies or speeds.

As is shown at block 504, a leading point of the blade may be pressed against the skin of a patient. In embodiments, prior to the pressing of the leading point of the blade against the skin of the patient, the user may pinch the skin of the patient. This may provide the user with a larger area in which the implantable device will be inserted. In addition, pinching the skin may provide a larger opening once the blade is inserted as a larger area of skin may contact the width of the blade. In embodiments, the user may press the leading point of the blade without pinching the skin of the patient as this may not be necessary to provide a desired contact area for the blade.

As is shown at block 506, the skin of the patient may be punctured by the leading point of the blade. In addition and as is shown at block 508, the blade may be extending into the patient such that sidewalls of the housing are against the skin of the patient. Extending the blade into the patient may include forming a pocket within the patient defined by a width of the blade. It may be beneficial to use a blade that has edges curved about the leading point such as shown in FIGS. 2C-E. The curved edges of the blade may effectively match the pinched surface of the skin, and provide a substantially horizontal cut in the skin of the patient. In addition, after the blade is extended into the patient, the blade may be removed, and an implantable medical device may be inserted via an introducer. Extension of the blade within the patient may define the pocket. Further, the pocket may be closed thereby sealing the implantable medical device within the patient. Closing the pocket may include sealing the implantable medical device within the patient by suturing or gluing the cut in the skin of the patient.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An apparatus comprising:
a blade comprising a leading point configured to puncture skin of a patient;
a housing comprising an opening through which the blade extends, the housing being configured to hold the blade and provide a surface for a user to control the housing and the blade, the opening comprising sidewalls configured to control a depth to which the blade extends under the skin of the patient; and
a motor arranged with the housing and configured to mechanically vibrate the blade.

2. The apparatus of claim 1, wherein the blade is symmetric about the leading point.

3. The apparatus of claim 2, wherein the blade comprises edges, symmetric about the leading point, configured to control a width at which the blade opens the skin of the patient.

4. The apparatus of claim 1, further comprising a power source to power the motor to mechanically vibrate the blade.

5. The apparatus of claim 4, wherein the power source is configured to activate in response to the user gripping the housing.

6. The apparatus of claim 1, wherein the motor is at least one of a piezoelectric motor, an electromagnetic vibratory motor, an electrostatic vibratory motor, and an electroactive polymer.

7. The apparatus of claim 1, further comprising at least one control mechanism configured to switch between at least two modes that provide the at least two levels of power to mechanically vibrate the blade.

8. A method comprising:
activating a motor, arranged with a housing, to mechanically vibrate a blade held within the housing;
pressing a leading point of the blade against skin of a patient;
puncturing the skin of the patient with the leading point of the blade; and
extending the blade into the patient until sidewalls of the housing are against the skin of the patient.

9. The method of claim 8, wherein the step of activating the motor comprises activating one of at least two modes to provide one of at least two levels of power to mechanically vibrate the blade, and wherein the step of extending the blade into the patient comprises forming a pocket within the patient defined by a width of the blade.

10. The method of claim 8, further comprising removing the blade and inserting an implantable medical device, via an introducer, within the pocket defined by insertion of the blade into the patient, and closing the pocket to seal the implantable medical device within the patient.

11. The method of claim 9, wherein the step of closing the pocket comprises sealing the implantable medical device within the patient by at least one of suturing and gluing a cut in the skin of the patient.

12. A system comprising:
an implantable medical device;
an implantation apparatus comprising:
a blade comprising a leading point configured to puncture skin of a patient,
a housing comprising an opening through which the blade extends, the housing being configured to hold the blade and provide a surface for a user to control the housing and the blade, the opening comprising sidewalls configured to control a depth to which the blade extends under the skin of the patient, and
a motor arranged with the housing and configured to mechanically vibrate the blade; and
an introducer configured to position the implantable medical device under the skin of the patient.

13. The system of claim 12, wherein the implantable medical device is at least one of an implantable loop recorder, a cardiac monitor, a pacemaker, and a defibrillator.

14. The system of claim 12, wherein the blade is symmetric about the leading point.

15. The system of claim 14, wherein the blade comprises edges, symmetric about the leading point, configured to control a width at which the blade opens the skin of the patient.

16. The system of claim 12, wherein the implantation apparatus further comprises at least one control mechanism configured to switch between at least two modes that provide the at least two levels of power to mechanically vibrate the blade.

17. The system of claim 16, further comprising a power source to power the motor to mechanically vibrate the blade.

18. The system of claim 17, wherein the power source is configured to activate in response to the user gripping the housing.

19. The system of claim 17, wherein the power source is configured to activate in response to the user pressing the at least one control mechanism.

20. The system of claim 12, wherein the motor is arranged within the housing, and the motor is directly coupled to the blade.
